(19) United States
(12) Patent Application Publication
(10) Pub. No.: US 2008/0285378 A1
(43) Pub. Date: Nov. 20, 2008

(54) Disposable Homogenizer Device and Its Use for the Preparation of Homogenized Samples

(76) Inventor: Gianmarco Roggero, Orbassano (Torino) (IT)

Correspondence Address: MERCHANT & GOULD PC, P.O. BOX 2903, MINNEAPOLIS, MN, 55402-0903, US

(21) Appl. No.: 11/666,576

(22) PCT Filed: Oct. 26, 2005
(86) PCT No.: PCT/EP2005/055565

§ 371 Date: Apr. 26, 2007

(30) Foreign Application Priority Data
Oct. 27, 2004 (IT) TO2004A000743

(51) Int. Cl. G01N 1/28; C12M 3/08

(52) U.S. Cl. 366/140

(57) ABSTRACT

A homogenizer device, is for the preparation of homogenized samples of food substances the bacterial flora content of which is to be analyzed includes a cup-like container (4) for receiving the sample to be homogenized and a lid (16) for the closure of the cup-like container. The homogenizer device (40, 42) includes a shaft (40) having a proximal end (44) provided with an engagement (44) for connection to a drive and a distal end (46) connected to an abrasive homogenizer member (42), the shaft (40) being mounted rotatably in an opening (24) of the lid (22, 16) and being provided with a tapered proximal portion (56) having a diameter which increases towards the proximal end (44) and which opposes forward movement of the shaft towards the sample by dimensional interference with the opening (24). Under the effect of a drive suitable for imparting to the shaft (40) a rotary movement and an axial thrust towards the sample, the shaft can bring about a progressive enlargement of the opening (24) such as to permit the progressive forward movement of the shaft towards the sample which movement is opposed by the increasing interference of the tapered portion (56) with the opening (24).

The present invention relates to a disposable homogenizer device, particularly for the preparation of homogenized samples of food products the bacterial flora content of which is to be analyzed.

The field of use of the device of the invention is, in particular, the quantitative analysis of the bacterial flora both of primary foods, that is, foods occurring naturally (such as vegetables, meats, fruit, etc.), and of derivatives, that is, food products resulting from a manual, culinary, or industrial process for the transformation of primary foods.

Worldwide, approximately 120 million analyses are currently performed on ingredients each year and the trend is clearly towards an increase dictated by the ever greater sensitization of consumers towards safe food products that are free of undesired bacteria.

However, no implements are commercially available which enable the homogenization of the sample to be automated. The implements used up to now can be found in systems which require the constant presence of an operator, for example:
a) high speed drills which are operated as required for each individual sample,
b) pestles which break up the sample in a mortar,
c) nylon envelopes which are struck violently by small hammers.

The use of conventional homogenizers is not desirable since, in order to analyze the bacterial flora, it is necessary to ensure the re-establishment of conditions of complete sterility for each sample.

An object of the present invention is to provide a disposable homogenizer device which enables the homogenization operation to be performed in a reproducible manner and which is suitable for the homogenization of any material, whether it be soft or extremely hard.

Another object of the invention is to provide a device which enables the homogenization operation to be performed without the constant presence of an operator.

Yet another object of the invention is to provide a homogenizer device which has a low production cost and which can therefore be implemented as a disposable device which can prevent risks of contamination of the sample undergoing analysis, avoiding the need for sterilization of the homogenizer device for further use thereof.

In view of these objects, the subject of the invention is a homogenizer device having the characteristics specified in the appended claims.

Further characteristics and advantages of the device according to the invention will become clear from the following detailed description with reference to the appended drawings which are provided by way of non-limiting example and in which:

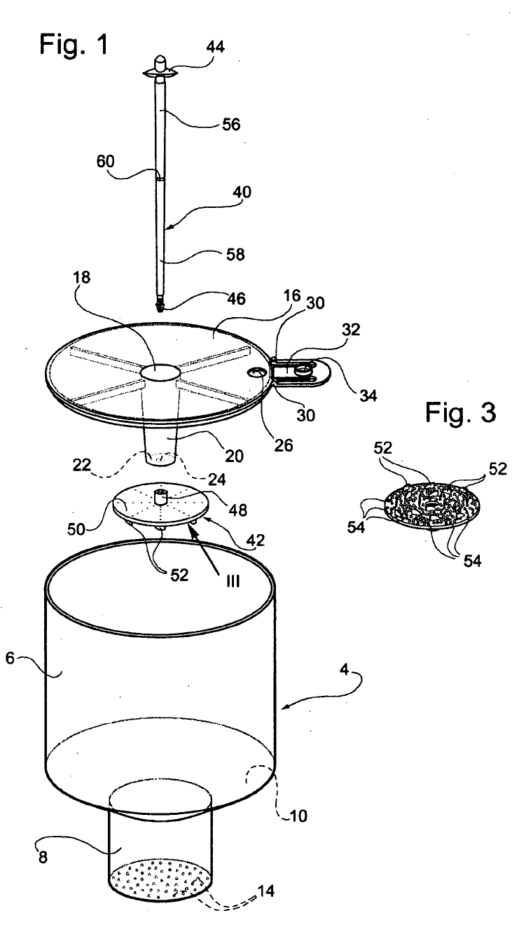

FIG. 1 is an exploded, perspective view of the homogenizer device according to the invention,

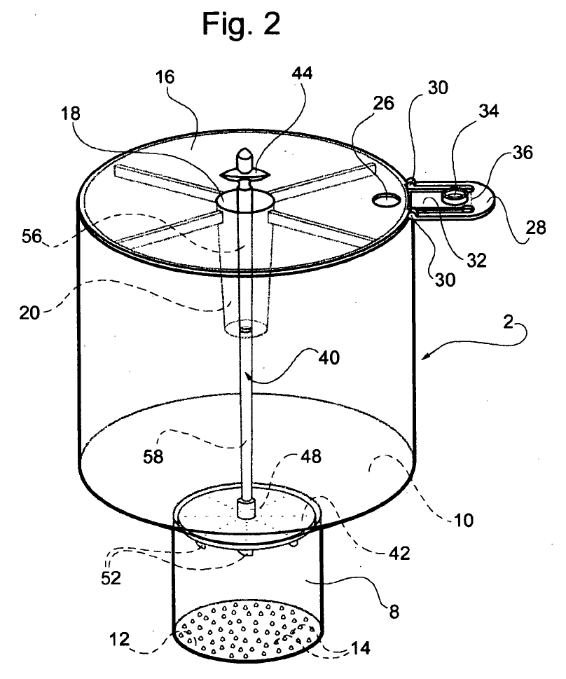

FIG. 2 is a perspective view of the device of FIG. 1 in the assembled configuration, and

FIG. 3 is a view of a detail of FIG. 1, taken on the arrow III.

The drawings show the homogenizer device, generally indicated 2, which is formed as a disposable device, that is, a device which is intended to be used only once for the homogenization of a sample and is therefore preferably made of low-cost plastics materials.

The device comprises a cup-like container 4 of plastics material, for example, transparent polyethylene, comprising an upper cylindrical portion 6 and a lower cylindrical portion 8 of smaller diameter which are connected to one another by a frustoconical intermediate portion 10.

The lower portion 8, which acts as a well, is intended to receive the sample to be homogenized and preferably has a base 12 which has a surface with irregular knurling or beading suitable for improving the efficacy of the homogenization, and/or which has a plurality of pointed elements 14 suitable for retaining the sample undergoing homogenization.

Typically, the well 8 has a capacity suitable for a sample of about 40 g whereas the wider, upper portion 4, which is intended to contain a homogenization buffer solution, may have a capacity of about 400 ml.

A lid 16 of plastics material, preferably transparent polyethylene, is associated with the cup-like container 4 and can engage the mouth of the container 4 in a leaktight manner.

In the embodiment shown, the lid 16 has a central opening 18 from the mouth of which there extends a tubular element 20 which is closed at the bottom by a base 22 having a circular opening 24.

The lid 16 preferably has a peripheral opening 26 for enabling a pipette to be introduced into the closed container for the withdrawal of the homogenized substance. A tongue-like element 28 is articulated to the lid at 30 and has a flexible gripping member 32 provided with a closure element 34 suitable for engaging the opening 26. The gripping member 32 has a free end 38 which is not connected to the rim of the lid and which faces towards the lid when the tongue 28 is in the open configuration; the gripping member 22 is articulated to the body of the tongue-like element by a film hinge 36.

To close the opening 26, the tongue-like element 28 is turned over onto the lid by pivoting of the gripping member 32 so as to bring the closure element 34 into engagement with the opening 26. Opening is performed by exerting a pull on the gripping member 32; the film hinge 36 may be of a size such that the opening operation leads to tearing thereof and the detachment of the gripping member from the tongue-like element so as to prevent re-use of the lid when the homogenization has been performed and after the sample has been withdrawn.

The homogenizer member of the device comprises a shaft 40 and an abrasive homogenizer disk 42. The shaft 40 has, at its proximal end, engagement means 44, for example, bayonet-type engagement means, arranged for engaging a drive transmission member forming part of a drive device with which the homogenizer device illustrated is intended to cooperate.

The distal end of the shaft 40 has engagement means, preferably of the snap type 46, suitable for a releasable connection to the disc 42. In the embodiment shown, the snap-engagement means 46 comprise two toothed prongs which snap engage in the internal cavity of a tubular portion 48 of the abrasive disk 42.

The disk 42, which is shown from above in FIGS. 1 and 2 and from below in FIG. 3, is constituted by a circular disk-shaped body which has a plurality of through-holes 50 (for example, with a diameter of about 200 microns), in a ray-like arrangement and which, as will be explained more fully below, enable the homogenized sample to be dispersed in the homogenization buffer by passing through the homogenizer disk as a result of the forward movement of the disk 42 towards the base of the well 8.

The size of the abrasive homogenizer disk 42 is such that it can rotate with limited clearance inside the well 8.

The lower surface of the abrasive homogenizer disk (FIG. 3) has a plurality of projecting blade-like elements 52, for example, arranged in concentric circles and spaced apart angularly and, preferably, a plurality of pointed projecting elements 54 with highly abrasive action.

The homogenizer disk 42 may be made of a very hard plastics material, for example, polyamide reinforced with glass or glass-fibre fillers (for example, nylon glass 30) but the use of metallic materials should not be excluded.

The shaft 40 has a distal portion 58 and a proximal portion 56 which are preferably separated by a groove 60. The radial dimension of the distal portion 58 is such that that portion can be inserted in the opening 24 of the base 22 of the tubular element 20. The proximal portion 56 is a conical portion having a diameter which increases progressively towards the proximal end of the shaft 40. Adjacent the groove 60, the tapered proximal portion 56 therefore has a minimum diameter which is greater than the diameter of the groove 60 and such as to interfere dimensionally with the diameter of the opening 24 so as normally to oppose and/or prevent the passage of the portion 56 through the opening 24. The distal portion 58 may be cylindrical, tapered, or even prismatic, preferably with a maximum radial dimension greater than the diameter of the groove 60 but in any case such as to permit the introduction of that portion into the opening 24.

The shaft 40 may be made of a very hard, rigid plastics material such as polyamide reinforced with glass-fibre fillers (nylon glass 30) or of metallic material and is in any case made of a material having a hardness and a wear resistance substantially greater than those of the material of the wall of the lid 16 (in the embodiment shown of the base 22) which defines the opening 24 in which the shaft is mounted rotatably.

In the assembled configuration of the homogenizer device, the shaft 40 is inserted in the opening 24 until the groove 60 is brought into the vicinity of the edges of the wall 22 delimitating that opening. In this position, the shaft is freely rotatable in the opening 24 and the portions 56 and 58, which have dimensions greater than the diameter of the groove, act as axial stops for the shaft. It will, however, be appreciated that the presence of a groove 60 is purely optional and preferred.

The disk 42 is then connected to the shaft 40 by engaging the engagement means 46 and 48 with one another. In the assembled condition of the device, the disk 42 is substantially flush with the mouth profile of the cylindrical portion or well 8 of the container 4.

The sample to be homogenized is inserted in the well 8; a buffer solution in which the homogenized sample is to be dispersed is then introduced into the container 4. The container 4 is then closed by means of the lid 16 with which the shaft 40 and the homogenizer disc 42 are associated in the manner indicated above. The homogenization operation is performed by connecting the proximal end of the shaft 40 to drive means which is arranged for rotating the shaft and for exerting on the shaft an axial thrust which tends to cause the shaft 40 and the abrasive disk 2 to advance towards the sample. By virtue of the difference in hardness between the material constituting the shaft and the material of the lid defining the opening 24, the rotary movement of the shaft brings about, by sliding friction, wear in the wall 22 and a progressive enlargement of the opening 24 such as to permit the progressive forward movement of the shaft towards the sample, which movement is opposed by the increasing interference of the diameter of the tapered portion 56 with the wall 22 delimiting the opening 24.

The progressive forward movement of the shaft and of the homogenizer disc 42 towards the sample is particularly advantageous for the homogenization of solid materials the consistency of which is such as further to oppose the forward movement of the disk. Moreover, owing to the enlargement of the opening 24 which is achieved upon completion of the homogenization process, the homogenizer device cannot generally be re-used for a further homogenization operation performed on another sample, thus ensuring that each homogenization operation performed with the device of the invention can be carried out in completely sterile conditions and without risk of contamination resulting from the re-use of the device.

In the course of the homogenization process, the homogenized sample contained in the well 8 emerges from the well through the holes 50 in the disc and is dispersed in the homogenization liquid as a result of the piston-like action of the disc 42 advancing into the well 8. This liquid can then easily be extracted from the container 4, for example, by means of a pipette inserted in the opening 26.

The homogenizer device is suitable for the homogenization both of food substances of firm consistency such as seeds, dried pulses, dried fruit, nuts and of relatively soft samples such as vegetables, fresh pasta and food fillings.

The homogenization time required varies according to the consistency of the sample and is generally within the range of from 30 samples/hour (for very firm products) to 480 samples/hour for softer products.

The homogenizer device is intended to be used in association with an apparatus which is automatic or can be automated and which is provided with drive means and drive transmission means that can engage the shaft 40 of the homogenizer device and impart to the shaft the high-speed rotary motion which is necessary for the homogenization and an axial thrust which is suitable for bringing about the translation of the shaft towards the sample. By way of example, this apparatus may comprise a homogenization station provided with the drive transmission means and a belt conveyor member which transports the homogenizer devices to the homogenization station. The homogenizer devices described above may advantageously be positioned in a support member provided with a plurality of cavities in which the wells 8 of the homogenizer devices are inserted. Support members with various characteristics (for example, of different colours) corresponding to different hardnesses of the material to be homogenized, may be provided. A conveyor member, for example, a belt conveyor, transports the support members to the homogenization station which may be provided with sensor members that indicate the presence of a support member and of the homogenizer devices so as to bring about, by means of a signal-processing unit, the descent of the drive transmission member into engagement with the shaft 40. The homogenization station may be provided with sensors which can determine the characteristics of the support member (for example, its colour) and can select the parameters of the homogenization process, such as the rate of rotation and the forward movement of the shaft, according to the type of material to be homogenized. Homogenization devices of the invention are therefore suitable for use in homogenization operations which can be completely automated and which are carried out with maximum operator safety, ensuring a repeatable and standardizable homogenization result with a substantial reduction in the manual labour required.

1. A homogenizer device, for the preparation of homogenized samples of food substances, bacterial flora content of which is to be analyzed, comprising:
a cup-like container for receiving a sample to be homogenized,
a lid for closure of the cup-like container, and
homogenizer means comprising a shaft having a proximal end provided with engagement means for connection to drive means and a distal end connected to an abrasive homogenizer member, the shaft being mounted rotatably in an opening of the lid and being provided with a tapered proximal portion having a diameter which increases towards the proximal end and which opposes forward movement of the shaft towards the sample by dimensional interference with the opening, wherein, under the effect of drive means suitable for imparting to the shaft a rotary movement and an axial thrust towards the sample, the shaft can bring about a progressive enlargement of the opening to permit the progressive forward movement of the shaft towards the sample, wherein movement is opposed by the increasing interference of the tapered portion with the opening.

2. A homogenizer device according to claim 1, wherein the opening is formed in a wall of the lid which is made of a material having a hardness and/or wear resistance substantially less than the hardness and/or wear resistance of the material comprising the tapered portion of the shaft.

3. A homogenizer device according to claim 1, wherein the lid has a central opening from which there extends towards the interior of the container a tubular element which has a base in which the opening is formed.

4. A homogenizer device according to claim 1, wherein the shaft has a groove interposed between a distal portion of the shaft, which can be introduced into the opening, and the tapered portion and in which, in the initial operative configuration of the device, the shaft is mounted to be freely rotatable in the lid with the wall delimiting the opening in the region of the groove.

5. A homogenizer device according to claim 1, wherein the abrasive homogenizer member comprises a disk-like body having a plurality of pointed projecting elements on its face which faces towards the interior of the container.

6. A homogenizer device according to claim 5, wherein the disk-like body has a plurality of blade-like projecting elements on its face which faces towards the interior of the container.

7. A homogenizer device according to claim 1, wherein the homogenizer member has dimensions such that it is rotatable with limited clearance inside a cylindrical end portion of the container and can translate axially in the manner of a piston in the end portion.

8. A homogenizer device according to claim 5, wherein the disk-like body has a plurality of through-holes such that the translation of the disk-like body towards the base of the container causes the homogenized sample to emerge through the holes.

9. A homogenizer device according to claim 1, wherein the cup-like container has a wider upper portion and a narrower end portion for receiving the sample.

10. A homogenizer device according to claim 1, wherein the container has a knurled or beaded base.

11. A homogenizer device according to claim 1, wherein the container has a base which is provided with a plurality of pointed elements for the retention of the sample.

12. (canceled)

13. A homogenizer device according to claim 1, wherein the abrasive homogenizer member is connected to the shaft by snap-engagement means.

14. A homogenizer device according to claim 1, wherein the lid has an opening and a tongue-like members which is articulated to the lid and is provided with a gripping member articulated to the tongue by a film hinge and having an element for the closure of the opening.

15. A homogenizer device according to claim 1, wherein the shaft and the abrasive homogenizer member are made of a plastics material having a hardness substantially greater than the hardness of plastics material comprising the lid.

16. A homogenizer device according to claim 15 wherein the shaft and/or the abrasive homogenizer element are made of a plastics material filled with abrasive fillers.

17. Homogenization apparatus comprising a homogenizer device according to claim 1 and drive means which can engage the shaft and impart to the shaft a rotary motion and an axial thrust towards the sample positioned in the container.

18. A Method of using a homogenizer device according to claim 1 comprising homogenizing food samples the bacterial flora content of which is to be analyzed.
